(19) United States
(12) Patent Application Publication
(10) Pub. No.: US 2025/0212901 A1
(43) Pub. Date: Jul. 3, 2025

(54) Method of Reduction and/or Elimination of a Target Agent

(71) Applicant: GEMINA I MAS D S.L., Jumilla (ES)

(72) Inventors: Pedro Simón Bernal, Jumilla (ES); Pedro José Simón García, Jumilla (ES); José Vilella Esplá, Jumilla (ES)

(21) Appl. No.: 18/852,798

(22) PCT Filed: Mar. 29, 2023
(86) PCT No.: PCT/ES2023/070204

§ 371 Date: Sep. 30, 2024

(30) Foreign Application Priority Data
Apr. 1, 2022 (ES) P202230297

(51) Int. Cl. A23B 2/46; A23B 2/00; A61L 2/04; A61L 2/07; A61L 2/24; G01K 13/02; G01K 13/024

(52) U.S. Cl. CPC A23B 2/46; A23B 2/003; A61L 2/04; A61L 2/07; A61L 2/24; G01K 13/024; G01K 13/026; A61L 2202/14

(57) ABSTRACT

The present invention relates to a method for reducing and/or eliminating a target agent, particularly from a fluid, preferably from a liquid food flow rate. Preferably, the present method makes it possible to sterilize and/or pasteurize a food in a liquid state, from the modification and control of the value of the case fatality rate Fi provided, by means of the control and regulation of process parameters Ti and Qi.

### CROSS-REFERENCE TO RELATED APPLICATIONS

This application is the United States national phase of International Application No. PCT/ES2023/070204 filed Mar. 29, 2023, and claims priority to Spanish Patent Application P202230297 filed Apr. 1, 2022, the disclosures of which are hereby incorporated by reference in their entireties.

### BACKGROUND OF THE INVENTION

### Field of the Invention

The present disclosure relates to a method for reducing and/or eliminating a target agent or safety target agent, particularly from a fluid, said fluid being pumpable, and said fluid preferably being a liquid food flow rate.

Preferably, the present method makes it possible to sterilize and/or pasteurize a food in a liquid state, to ensure the safety of the food during its processing, reducing and/or eliminating the target agent present in said liquid food flow rate based on the control (that is, recording, monitoring and verification) of a control measure, in addition to the modification of the parameters that define said sterilization and/or pasteurization process. The control measure of a heat treatment, particularly a pasteurization and/or sterilization process, is referred to as the set of specific combinations “Treatment temperature-treatment time” which is applied to the liquid food flow rate, characterized by a single parameter, referred to as the cumulative case fatality rate or F value or P value.

The present disclosure, therefore, relates to the regulation and control of the microbial load, enzymatic load, or of any other biochemical or chemical load, of the pumpable fluid through a heat treatment, preferably a pasteurization and/or sterilization process, carried out on the liquid food flow rate. That is, the present method carries out regulation and control of the values of the load level of the target agent present in said liquid food flow rate, thus ensuring that the target value (n_{threshold}) of the target agent, depending on the heat treatment applied, said target value (n_{threshold}) being the specific threshold number of log-decimal reductions in the initial load of the target agent, or safety target agent, present in the food.

### Description of Related Art

In the state of the art, there is extensive knowledge about heat treatments in food flow rates, such as pasteurization treatments, and systems that allow the application of said treatments, based on a holding time and temperature of the liquid food flow rate sufficient for said treatment to provide a case fatality rate of the target agent which meets the objective of heat treatment, particularly pasteurization and/or sterilization.

However, the methods known today carry out the heat treatment at issue based on preset and constant values of holding time and holding temperature, without knowing at each instant throughout the process the cumulative case fatality rate provided by said heat treatment, that is, the cumulative case fatality rate supplied by the system that applies said treatment at each instant, by means of the treatment temperature-time combination applied.

Due to oscillations in the holding temperature and flow rate to which the system for applying the heat treatment is subjected, it is not possible to know exactly and in real time the cumulative case fatality rate provided by said system against the target agent present in the liquid food flow rate for each applied treatment temperature-time combination recorded at each instant.

That is, the equipment that makes it possible to carry out heat treatments such as those indicated do not have automated systems that make it possible to reliably warn whether or not the combination of the holding temperature and/or flow rate values (related to the holding time) of said treatment provides a sufficient case fatality rate against the target agent that is intended to be eliminated or reduced, in order to exceed or not exceed the preset limit threshold value of said target agent, as the heat treatment is applied.

In this way, in the current equipment for applying heat treatments, such as pasteurization and/or sterilization, the efficacy of this treatment is unknown when the liquid food flow rate and the holding temperature vary and, therefore, it is not possible to regulate the parameters of the treatment in order to obtain the required efficacy.

Moreover, the lack of control of the efficacy of the heat treatment carried out on the liquid food flow rate implies that, although the treatment does not obtain the required values of the case fatality rate of the target agent, the liquid food flow rate leaves the heat treatment under certain inadequate conditions, without the possibility of modification and resulting in a waste of product by not meeting the predetermined objectives of safety or quality in food.

This implies, therefore, that currently known methods allow the application of a heat treatment to a fluid flow rate in order to try to reduce the volume of the microbial, enzymatic, biochemical or chemical load, of a target agent present or potentially present in the fluid, without the possibility of knowing the efficacy of said heat treatment against the target agent, based on the variation during the process of applying said treatment of the different temperature and flow combinations recorded at each instant. Therefore, it is not possible to know the real efficacy of a treatment of this type at each instant.

### SUMMARY OF THE INVENTION

The present disclosure makes it possible to solve the aforementioned problems by means of a process for the reduction and/or elimination of a target agent from a fluid by means of a system that allows the application of said process.

A first inventive aspect therefore relates to a method for reducing and/or eliminating a target agent from a fluid by means of a system comprising:
a tube extending between a first end and a second end, forming an internal volume V according to a path,
wherein the first end is a fluid inlet and the second end is a fluid outlet,
wherein the fluid enters the internal volume V of the tube through the inlet of the tube and runs along the path to the outlet of the tube, wherein said fluid is driven by a pump,
heating means fluidically connected to the inlet of the tube,
at least one fluid flow rate sensor, configured to measure the flow rate Q of the fluid driven by the pump,
at least one temperature sensor, located at the first and/or second end of the tube, configured to measure the temperature T of the fluid driven by the pump,
control means, comprising a controller and storage means,
the method comprising the following steps:
a) providing the storage means of the control means with the following predetermined values:
   t_{validated}, holding time of the fluid in the internal volume V of the tube, from its inlet to its outlet,
   T_{validated}, temperature of the fluid at the outlet of the tube,
   F_{target}, value of the case fatality rate required to achieve the required reduction and/or elimination of the target agent,
b) introducing and circulating by means of the pump, during the holding time t_{validated}, the fluid at the temperature T_{validated} in the interior volume V of the tube,
c) recording, by means of the at least one temperature sensor and the at least one flow rate sensor, for each instant of time tᵢ, ∀i=0, 1, . . . , the real temperature value of the fluid, Tᵢ and the real flow rate value Qᵢ, of the fluid in the interior volume V of the tube,
d) calculating for each instant of time tᵢ, using the control means and based on the records of the flow value Qᵢ of the fluid, the real holding time tₘᵢ of the fluid obtained in the interior volume V of the tube, wherein:

$${t}_{mi} = \frac{V}{{Q}_{i}}$$

e) calculating for each instant of time tᵢ, using the control means, the value of the case fatality rate provided by the system for the reduction and/or elimination of the target agent, Fᵢ, according to the values recorded and calculated in steps c) and d), by means of:

$${F}_{i} = {\int }_{{t}_{0}}^{{t}_{mi}} {10}^{( \frac{{T}_{i} - {T}_{ref}}{Z} )} dt ,$$

wherein Z is a characteristic parameter of the target agent, particularly a kinetic property of reduction and/or elimination of the target agent, and T_{ref} is the chosen reference temperature to express the case fatality rate provided, Fᵢ,
f) verifying for each instant tᵢ, using the control means, if condition Fᵢ<F_{target} is met, and
g) regulating, using the control means, if the condition of step f) is met, the temperature value of the fluid Tᵢ and/or the flow rate value of the fluid Qᵢ in the interior volume V of the tube, which verify the condition Fᵢ≥F_{target}.

In this way, the application of the present process on the target agent to be reduced and/or eliminated, present or potentially present in a fluid, is carried out through a system defined by means of a tube. In a particular embodiment, the tube is a continuous element, preferably manufactured in a single piece. In a particular embodiment, the tube is a continuous element, obtained by means of attaching a plurality of elements together, forming a tube once attached. Preferably, the elements are straight or curved tubes attached to each other consecutively by attachment means, preferably welding or mechanical couplings.

In a particular embodiment, said target agent is any microbial, enzymatic or biochemical agent, particularly, any microorganism present in the fluid that requires population control. In this way, any microbial, enzymatic or biochemical agent, particularly, any pathogenic or quality-altering microorganism that is more heat-resistant to the flora present or potentially present in food and can develop or survive in it, is referred to as a target agent, safety target agent or quality target agent.

The tube defines a path for the fluid, comprised between a first end and a second end of the tube. Said path is defined based on multiple configurations, the preferred configuration being a series of long, straight and parallel pipes, attached to one another by 180° C. bends.

The fluid therefore travels through the internal volume V defined by said tube, according to the path defined by it, entering the internal volume V through the inlet present at the first end, and leaving the interior of the tube through the outlet present at the second end of the tube. A pump, and particularly the drive power of said pump, allows the fluid to run along the path defined in the interior of the tube, said fluid therefore being pumped along its entire path.

The tube comprises a continuous section and a length, which defines its internal volume. V. In a particular embodiment, the section of the tube is constant.

Additionally, the system also comprises at least one fluid flow rate sensor, configured to measure the flow rate Q of the fluid driven by the pump within the internal volume V of the tube. In a particular embodiment, the at least one flow rate sensor is a flow meter that allows the fluid flow rate circulating through the tube to be measured.

Additionally, the system also comprises heating means fluidically connected to the tube, so that the fluid is heated prior to being introduced into the tube through the inlet present at the first end of said tube. Thus, the system raises the temperature of the fluid prior to its entry into the interior of the tube, and holds said temperature for a specific time within said tube, which provides the process with an efficacy in the objective of reducing and/or eliminating the target agent. In a particular embodiment, the outlet of the food flow rate from the heating means is connected in series with the inlet of the tube.

In a particular embodiment, the temperature of the fluid is raised in the heating means through steam injection or by indirect contact with any other hot fluid, through a heat exchanger.

Moreover, the system in turn comprises at least one temperature sensor, configured to record the temperature Tᵢ of the fluid in an instant of time tᵢ, ∀i=0, 1, . . . , m along its path through the internal volume V of the tube. In this way, the present method provides a single record of the temperature Tᵢ of the fluid in an instant of time t; for calculating the case fatality rate provided Fᵢ.

The at least one sensor that provides said record Tᵢ it is located at any intermediate point of the path of the fluid, located between the first and the second end of the tube, or at the inlet and/or outlet of the fluid in said tube, that is, at the first and/or second end of the tube, respectively. In a particular embodiment, the at least one temperature sensor is a probe, which takes a direct measurement of the temperature of the fluid at the point where it is located.

In a particular embodiment, for the calculation of Fᵢ carried out in step e) at each instant of time tᵢ, this method preferably uses the temperature record Tᵢ coming from the probe located at the outlet of the tube, that is, at the second end of the tube. Advantageously, this temperature record is the most conservative, since the outlet of the tube is the most unfavorable point due to energy losses into the environment, as the fluid circulates through the tube from the inlet to the outlet.

In a particular embodiment, the real temperature value of the fluid Tᵢ recorded in step c) is a constant value at any instant of time tᵢ. That is, the recorded temperature of the fluid particle entering the tube or exiting the tube remains constant as the fluid moves through the tube, regardless of whether the temperature sensor is located at the beginning or end of the tube. Thus, the system assumes that the temperature measured at the outlet of the tube has remained constant along its path through the tube, that is, from the inlet of the fluid in the tube to the outlet of the fluid in the tube, where the temperature Tᵢ recorded at the outlet of the tube is the temperature of the fluid along its path through the tube.

In this way, when the system has a single probe located at the outlet of the tube, the record of the temperature Tᵢ at any instant of time tᵢ will coincide with the real temperature of the fluid at the outlet of the tube. However, when the system does not have a temperature probe located at the outlet of the tube, but rather at the inlet or at any intermediate point between the inlet and outlet of the tube, the record of the temperature Tᵢ is considered to be the record coming from the probe located at the first end of the tube or at an intermediate point, as if it had been made at the second end of the tube, therefore considering the temperature Tᵢ at any instant of time tᵢ to be constant as the fluid circulates in the interior of the tube, that is, from the inlet of the fluid in the tube to the outlet of the fluid in the tube.

Finally, the system also comprises control means, said control means in turn including a controller configured to execute the control and regulation of the process, as well as storage means, configured to store data recorded by the at least one temperature sensor, as well as data related to the fluid present in the internal volume of the tube.

In a particular embodiment, the control means are configured to control the temperature of the fluid in the internal volume of the tube, as well as the holding time of the fluid in the internal volume of said tube, that is, the time it takes the fluid to travel through the entire internal volume V of the tube.

In a particular embodiment, the tube defines an internal volume V which, together with the fluid flow rate Q circulating through said internal volume V, makes it possible to obtain the minimum time necessary tₘ for a fluid particle to meet the target reductions n_{threshold} running along the entire internal volume V of the tube, where the minimum time necessary tₘ is the time calculated based on the fluid flow rate measured at the outlet of the tube.

The mentioned system makes it possible to carry out the method object of the first inventive aspect. Said process begins by incorporating a series of predetermined values into the storage means. These predetermined values are setpoint values, that is, working values of the system that have previously been determined in order to establish the treatment to be applied to the fluid by the system. Said previously calculated values are incorporated into the system as working setpoints to be followed by the system. The predetermined values to be provided to the system to begin its operation are the following:
t_{validated}: the holding time of the fluid in the internal volume V of the tube, from the inlet to the outlet of the fluid inside said tube. That is, the time it takes the fluid to run along the entire path defined by the tube. Said holding time is the minimum time established for the heat treatment carried out on the fluid housed in the internal volume V of the tube to be effective. This holding time is introduced into the system indirectly by means of the flow rate, taking into account the volume of the tube.
T_{validated}: the temperature of the fluid at the outlet of the internal volume V of the tube. That is, the outlet temperature of the fluid from inside the tube, after being subjected to the heating means, that is, after being subjected to the previous section of the system that allows the fluid to be heated up to the temperature required for the heat treatment carried out on the fluid to be effective.
F_{target}: value of the target or required case fatality rate to eliminate and/or reduce the target agent.

Once the previous predetermined parameters have been defined, the fluid is introduced into the interior volume V of the tube, and its circulation is caused by the pump, during the predetermined holding time t_{validated} at the predetermined temperature T_{validated}.

Subsequently, the temperature of the fluid Tᵢ and the value of the flow rate volume Qᵢ for an instant of time tᵢ, ∀i=0, 1, . . . , m are recorded on the fluid introduced into the tube through the at least one temperature sensor and the at least one flow rate sensor, where the recording point and therefore the location point of the at least one temperature sensor and of the at least one flow rate sensor is any point located between the inlet of the fluid into the interior of the tube until its outlet from the interior of said tube at its second end.

That is, the process records in the storage means the value of the temperature Tᵢ and the flow rate Qᵢ for an instant of time tᵢ, ∀i=0, 1, . . . , m, where t₀ is the instant of time in which the fluid enters the interior of the tube and tₘᵢ is the holding time, or instant in which the fluid exits from the interior of the tube.

In a particular embodiment, the value of the temperature Tᵢ recorded is Tₘ, that is, the temperature at the instant of time tₘ when the fluid exits from the interior of the tube.

In a particular embodiment, the process records in the storage means the value of various temperatures Tᵢ and various flow rate values Qᵢ for each instant of time tᵢ, ∀i=0, 1, . . . , m. Advantageously, this makes it possible to have a greater number of real data about the conditions of the treatment applied to the fluid, and therefore its efficacy. Through the records taken and the predetermined values, the present method makes it possible to calculate, using the control means and based on the temperature records Tᵢ and the flow rate value Qᵢ of the fluid, the holding time tₘᵢ of the fluid in the interior volume V of the tube, wherein:

$${t}_{mi} = \frac{V}{{Q}_{i}}$$

That is, at each instant of time tᵢ in which a record is made, the holding time is obtained tₘᵢ based on the flow rate Qᵢ, and the temperature Tᵢ of each particle of the fluid that leaves the second end of the tube in that instant.

In a particular embodiment, step d) further comprises calculating the holding time tₘᵢ factored by a magnification factor FM considering the circulation regime of the fluid. Said magnification factor FM depends on the factored speed of the fluid along the tube, thus establishing a conservative criterion for determining the heat treatment to be applied.

Likewise, this method makes it possible to calculate, using the control means, the value of the case fatality rate provided Fᵢ against the target agent at each instant of time tᵢ, which is defined by means of:

$${F}_{i} = {\int }_{{t}_{0}}^{{t}_{mi}} {10}^{( \frac{{T}_{i} - {T}_{ref}}{Z} )} dt ,$$

wherein Z is one of the kinetic properties of reduction and/or elimination of the target agent and T_{ref} is the chosen reference temperature with respect to which the case fatality rate, Fᵢ, is to be expressed. That is, the reference temperature T_{ref} is the temperature used as a reference for the comparison of its heat treatment with the heat treatment carried out by the present process.

In this way, a target agent having kinetic parameters D_{TREF} and Z (where D_{TREF} and Z are specific parameters of the target agent) is considered.

Moreover, a reference treatment with a unitary value is considered and, with said unitary reference treatment preferably having a duration (holding time) of 1 minute, it is used to compare the current heat treatment.

To define said reference treatment, preferably of a unitary value, this being 1 minute, the kinetic parameters D_{TREF} and Z are considered (where D_{TREF} and Z are, in this case, specific values of this reference agent that has served to define the reference unitary treatment).

In a particular embodiment, step e) further comprises calculating the case fatality rate provided against the reference agent, Fᵢ′.

$${F}_{i}^{′} = {\int }_{{t}_{0}}^{{t}_{mi}} {10}^{( \frac{{T}_{i} - {T}_{ref}}{Z} )} dt ,$$

wherein Z is a specific value of the reference agent used to define the reference treatment and T_{ref} is the chosen reference temperature with respect to which the case fatality rate, Fᵢ′, is to be expressed, which in this method coincides with the reference temperature chosen to define the case fatality rate Fᵢ. That is, T_{ref} is constant for the calculation of both Fᵢ′ and Fᵢ′.

The system makes a distinction between the target agent and reference agent. In this way, the target agent indicates that agent, preferably a pathogenic or quality-altering microorganism, against which it is desired to validate the heat treatment carried out by the present process with respect to the safety or quality of the processed fluid, particularly processed food. On the other hand, the reference agent is that agent, fictitious or real, from which its kinetic values are taken, particularly its reference thermal destruction kinetic parameters, D_{Tref} and Z.

The case fatality rate F is also technically known by other names such as: Sterilizing value, fatality at T_{ref.}, F_{Tref}^{Zref} or P_{Tref}^{Zref}.

In a particular embodiment, the cumulative case fatality rates Fᵢ are used as indicators of the degree of pasteurisation/sterilisation supplied by the system to the fluid, particularly a food, through the heat treatment applied by the present process.

By way of example, the cumulative case fatality rate known in the industry as F₀=F_{121.1° C.}^{10.0° C.} indicates the reference treatment applied to the sporulated form of a generic serotype of the bacterium Clostridium botulinum. The kinetic value Z for the sporulated form of this bacterium, which serves to define the cumulative case fatality index F₀, is 10° C. Considering a reference temperature T_{ref.}=121.1° C., the decimal reduction time D_{Tref.} is 0.25 minutes, which consequently corresponds to a reference treatment of: F_{121.1° C.}^{10.0° C.=F}₀=3 minutes at 121.1° C. to achieve 12 log₁₀ reductions of the reference agent.

In a particular embodiment, in step e) the target agent and the reference agent may coincide and, accordingly, the values of the case fatality rates will also coincide, that is Fᵢ=F′ᵢ.

Once the value of the case fatality rate provided against the target agent Fᵢ at the instant of time t has been calculated, it is compared with the value of the target case fatality rate that is required, predetermined and entered in the first step, F_{target}, so that the present method makes it possible to evaluate if the treatment applied to the fluid is correct and effective at the instant of time tᵢ.

In a particular embodiment, the present process compares, for each instant of time tᵢ in which a fluid temperature record Tᵢ has been made, the calculated value of the case fatality rate provided against the target agent Fᵢ in the instant of time tᵢ with the value of the target case fatality rate that is required, predetermined and entered in the first step, F_{target}, so that the present method makes it possible to evaluate if the treatment applied to the fluid is correct and effective at the instant of time tᵢ.

In this way, step f) of the present method verifies, using the control means, if Fᵢ<F_{target}, that is, if the value of the case fatality rate provided by the system by means of the heat treatment applied at the instant of time tᵢ exceeds the value of the case fatality rate F_{target} for the control of the fatality supplied against the target agent.

If the condition Fᵢ<F_{target} is verified, then the treatment applied to the fluid at the instant of time tᵢ is not considered effective, as the value of the case fatality rate provided Fᵢ is lower than the value of the minimum case fatality rate F_{target} determined that is required, preferably previously validated and, therefore, required for adequate fluid treatment.

In this case, the present method carries out step g), whereby the necessary parameters, in this case the temperature value of the fluid Tᵢ and/or flow rate value Qᵢ of the fluid in the interior volume V of the tube, are regulated in order to verify the condition Fᵢ≥F_{target}.

Advantageously, the present method allows a control and regulation of the treatment applied to the fluid, checking the efficacy of the treatment and the scope of the target agent at each instant. That is, the present method optimizes the process of reducing and/or eliminating a target agent in the fluid, regulating the operation of the system based on the joint regulation of the holding time and the holding temperature, as an indirect regulation of the value of the case fatality rate provided by the system Fᵢ.

In a particular embodiment, step g) of the present method is carried out by means of a PID algorithm of the controller of the control means.

That is, the PID algorithm allows the control of the system through the control of the established parameters. Said PID algorithm is integrated in the control means of the system, and allows the regulation of the temperature of the fluid Tᵢ, among other possible parameters such as flow rate Qᵢ.

In a particular embodiment, the PID algorithm receives, as part of the parameters to be controlled, the value of the target or required case fatality rate, F_{target}, additionally calculating the value of the case fatality rate provided, Fᵢ, in the recorded instant(s) and comparing the values in order to establish if the treatment carried out on the fluid is adequate.

In a particular embodiment, in which the value of the temperature of the fluid Tᵢ is constant during the treatment holding time of the fluid in the tube, step d) of the present method comprises calculating the holding time tₘᵢ of the fluid in the interior volume V of the tube, where:

$${t}_{mi} = \frac{{V}_{M}}{{Q}_{i}}$$

Said calculation of the holding time tₘᵢ is increased in this particular embodiment by a magnification factor FM considering the food circulation regime, thus establishing a conservative criterion for determining the heat treatment to be applied.

In the present particular embodiment, the fastest food particle is considered for each of the records of the flow rate value Q; for each instant of time tᵢ.

In this way, in the present particular embodiment, the fluid regime is obtained from the mean circulation speed of the fluid in the tube and the calculation of the Reynolds number (Re), in turn obtained based on the viscosity and density data of the fluid flowing through the tube.

In the present particular embodiment, when Re<4000, it is considered that the fluid circulates in a laminar regime, while when Re≥4000, it is considered that the fluid circulates in a turbulent regime.

Additionally, based on the previously calculated Reynolds number (Re), the flow behavior index (n) of the fluid (characteristic parameter of the fluid and therefore known), as well as the density and viscosity of said fluid, the magnification factor FM of the already calculated mean circulation speed is obtained. This magnification factor FM makes it possible to calculate the factored speed as follows:
For 0<Re<4000 (laminar flow), the following is met:

$$Newtonian fluids ( n = 1 ) ; FM = \frac{{v}_{max}}{{v}_{m e a n}} = 2$$

$$Non - Newtonian fluids ( n \neq 1 ) ; FM = \frac{{v}_{max}}{{v}_{m e a n}} = \frac{3 n + 1}{n + 1}$$

For 4000≤Re≤35000 (turbulent flow), the following is met:

$$Newtonian fluids ( n = 1 ) ; FM = \frac{{v}_{max}}{{v}_{m e a n}} = \frac{f ( R e )}{{v}_{m e a n}} ; \text{} where f ( Re ) = \frac{\frac{1}{( 0.711 - 0.05975 log {( Re )}^{2}}}{1 - 0.09171 log {( Re )}^{2} + 0.0 \; 0059 log {( Re )}^{4}}$$

For Re>35000 (turbulent flow), the following is met:

$$Newtonian fluids ( n = 1 ) ; FM = \frac{{v}_{max}}{{v}_{m e a n}} = \frac{\frac{1}{0.0336 {log}_{10} {v}_{m e a n} + 0.662}}{{v}_{m e a n}}$$

Likewise, the calculation ratio of step e), relating to the value of the case fatality rate provided, Fᵢ, is obtained as follows:

$${F}_{i} = {10}^{( \frac{{T}_{i} - {T}_{ref}}{Z} )} {\int }_{{t}_{0}}^{{t}_{mi}} dt$$

In a particular embodiment, the present method comprises a validation step a.1) for validating the values of step a). Said validation step advantageously makes it possible to establish the working conditions of the present process.

That is, established during the validation step are the following conditions or predetermined and necessary values of the following variables:
t_{validated}: the holding time of the fluid.
T_{validated}: the temperature of the fluid at the outlet of the tube.
F_{target}: the value of the required target case fatality rate.

In validation step a.1), these values are obtained through an inference process in a validation graph, wherein the validation of the control measure is carried out, said control measure being the cumulative case fatality rate, making it possible to deductively determine the predetermined values, or setpoint values, of temperature T_{validated} and flow rate volume or holding time t_{validated} which ensure that the acceptable threshold (n_{threshold}) of log₁₀ reductions of count n of the target safety agent (target agent) is equaled or exceeded, thus defining the required target case fatality rate F_{target}.

In a particular embodiment, the validation graph allows the modification and regulation of five parameters at will of the user, wherein each modification is shown in the validation graph, as is its influence over the other parameters and over the achievement of the target threshold of log₁₀ reductions of safety or quality count n to be achieved (n_{threshold}) in order to validate the process. The parameters that allow modification are:
Holding temperature at the outlet of the tube.
Holding time in the tube considering the fastest particle of the fluid, particularly of the food, (PMRA),
Fluid flow rate,
Initial target agent population in the food before heat treatment.
Efficacy of the treatment against the target agent (n_{.target}), wherein n_{target} are the decimal logarithmic reductions obtained against the target agent by the temperature-time combination chosen in the validation process.

In a particular embodiment, validation step a.1) of the treatment carried out by the present process, comprises graphically inferring the holding temperature and holding time values that make it possible to achieve that the cumulative case fatality rate provided by the system that carries out the present process on the fluid equals or exceeds the fatality value required to achieve that the efficacy of the process (n_{target}) reaches the value n_{threshold} established as an objective to guarantee the safety or quality of the fluid, particularly of the processed food.

In this way, the system receives to carry out step a) of the process, the holding temperature value T_{validated} and holding time value t_{validated} of the fluid already validated during this step a.1), in addition to the value of the required target case fatality rate F_{target} using these values in steps f) and g) of the process.

In a particular embodiment, the verification step f) can be performed graphically, using a graph similar to the validation graph, which also represents the values for each instant of time tᵢ of the case fatality rates provided at the reference temperature against the target agent and reference agent and the holding temperature and holding time records recorded at each instant of time tᵢ.

In a particular embodiment, the present method comprises the previous step of sterilizing the tube of the system, as well as possible additional cooling means for the fluid that are connected after the tube, that is, fluidically connected to the outlet of the tube, reducing and/or eliminating a target agent present in said tube and/or in said additional cooling means, by means of a sterilization fluid, preferably by means of steam or hot water.

Advantageously, this allows the sterilization of the internal volume of the system and of the tube through which the fluid circulates during the treatment. Additionally, it also allows the sterilization of additional cooling means which are fluidically connected to the outlet of the tube.

In a particular embodiment, the previous step of sterilizing the tube comprises the objective of maintaining the system above a preset threshold temperature in a determined temperature probe for the time strictly necessary to reach a specific value of the preset cumulative case fatality rate F₀.

Thus, in the present particular embodiment, the previous sterilization step is performed by calculating the cumulative case fatality rate F₀, using as a temperature probe any one belonging to the tube. This temperature probe is the one that provides the records Tᵢ for calculating the cumulative case fatality rate F₀:

$${F}_{0} = {\int }_{{t}_{0}}^{{t}_{m i}} 1 {0}^{( \frac{{T}_{i} - {T}_{ref}}{Z} )} dt ,$$

wherein Z=10° C. is a characteristic kinetic parameter of the microorganism Clostridium botulinum and T_{ref}=121.1° C., the chosen reference temperature to express the case fatality rate of the reference treatment.

In a particular embodiment, the temperature sensor can be located in cooling means connected after the tube, that is, fluidically connected to the outlet of the tube.

In this case, the system performs the verification of compliance with the objective of reaching the preset value F₀ to complete sterilization of either the tube of the system, or the tube and the cooling means of the system, and calculates the time elapsed to reach the cumulative case fatality rate F₀. In a particular embodiment, the value of the cumulative case fatality rate F₀ reached during the sterilization of the equipment is obtained by numerical integration according to the trapezoidal method.

In a particular embodiment, the previous step of sterilizing the tube comprises a first step of incorporating a series of predetermined values into the storage means of the control means.

These predetermined values are setpoint values, that is, working values of the system that have previously been determined in order to establish the sterilization treatment to be applied. Said previously calculated values are incorporated into the system as working setpoints to be followed by the system, where these values are:
F_{validated sterilization}: Value of the target sterilization index, that is, the value of the target case fatality rate, obtained from the data of the target agent considered to previously sterilize the equipment. In this way, the minimum case fatality rate to be achieved, either in the tube and/or in the cooling means located behind the tube, is established.
T_{start sterilization}: temperature value in at least one given temperature sensor from which the cumulative sterilization index is calculated. Said temperature sensor can be located, as previously indicated, either in the tube and/or in the cooling means located after the tube, where the point at which said sensor is located is the temperature recording point.

Once the above predetermined parameters have been defined, the sterilizing fluid, particularly steam or hot water, is introduced into the interior volume V of the tube and circulated by means of the pump. It is necessary for said sterilising fluid to be at the predetermined temperature T_{start sterilization}, so that the temperature of said sterilizing fluid is controlled and regulated until reaching the indicated value of the temperature T_{start sterilization}. Only from the instant in which the sterilizing fluid has reached the mentioned value, is the value of the cumulative case fatality rate F₀ calculated. That is, it is not possible to start recording the temperatures of the sterilizing fluid until the temperature of said fluid reaches the predetermined value in the previous step.

Subsequent, the temperature of the fluid Tᵢ for an instant of time tᵢ, ∀i=0, 1, . . . , m is recorded on the fluid introduced into the tube, through at least one temperature sensor. In particular, the recording point of said temperature of the fluid Tᵢ is the location point of the at least one temperature sensor by means of which said recording is made, the at least one temperature sensor being located either at any point between the inlet of the fluid into the tube to its outlet from the interior of said tube at its second end or in the cooling means located at the outlet of the tube, that is, in the cooling means after the tube. In particular, t₀ is the instant of time in which the temperature of the sterilizing fluid is the temperature T_{start sterilization}. Additionally, tₘ is the instant of time in which the cumulative case fatality rate equals the value of the target case fatality rate, that is, in which F₀=F_{validated sterilisation}.

The sterilization process then stores the temperature value Tᵢ in the storage means for each instant of time tᵢ. Based on previous records, sterilisation requires calculating the cumulative case fatality rate F₀ according to the ratio:

$${F}_{0} = {\int }_{{t}_{0}}^{{t}_{m i}} 1 {0}^{( \frac{{T}_{i} - {T}_{ref}}{Z} )} dt$$

Wherein Z=10° C. is a characteristic kinetic parameter of the microorganism Clostridium botulinum and T_{ref}=121.1° C., the chosen reference temperature to express the case fatality rate of the reference treatment.

Finally, the present sterilization step determines if the value of the cumulative case fatality rate F₀ and the value of the validated sterilization index F_{validated sterilization}, meet the ratio F₀>F_{validated sterilization}, that is, the cumulative case fatality rate is higher than the value of the target sterilization index. In the event that the present ration is met, it is considered that the sterilization step provides a satisfactory result, so it is considered that the system has a sufficient level of sterility to start the step of applying heat treatment to the fluid, particularly, to the liquid food flow rate.

In a particular embodiment, the system further comprises a bypass valve, and wherein step g) of the method further comprises controlling the actuation of said bypass valve, particularly, acting on the opening and/or closing of the bypass valve.

In this way, the bypass valve allows diverting the fluid flow rate to prevent said fluid, particularly a food, from entering the packaging systems after the heat treatment provided in the tube, in the event that the fatality provided by the heat treatment has not been sufficient. This bypass valve can be located at the inlet or outlet of the tube.

In a second inventive aspect, the present disclosure defines a computer program configured to implement the method according to the first inventive aspect.

### BRIEF DESCRIPTION OF THE DRAWINGS

To complete the description and for the purpose of aiding to better understand the features of the present disclosure, a set of figures is attached to the present specification as an integral part thereof, in which the following is depicted in an illustrative and non-limiting manner:

FIG. 1 shows a flowchart of a particular embodiment of a pasteurizing/sterilizing equipment for the application of a method for reducing and/or eliminating a target agent from a fluid, comprising a bypass valve located at the first end of the tube.

FIG. 2 shows a flowchart of a particular embodiment of the pasteurizing/sterilizing equipment for the application of a method for reducing and/or eliminating a target agent from a fluid, comprising a bypass valve located at the second end of the tube.

FIG. 3 shows a particular embodiment of a validation graph, with the process pending validation, that is, with the value of the case fatality rate provided by the current treatment lower than the value of the required target case fatality rate.

FIG. 4 shows the validation graph of FIG. 3 with the process validated, that is, with the value of the case fatality rate provided by the current treatment equal to the value of the required target case fatality rate.

FIG. 5 shows a particular embodiment of a graphic verification and control process of the system of FIGS. 1 and 2.

FIG. 6 shows a particular embodiment of a graphic verification and control process of the system of FIGS. 1 and 2.

FIG. 7 shows a particular embodiment of a graphic verification and control process of the system of FIGS. 1 and 2.

FIG. 8 shows a particular embodiment of the graph of the sterilization process prior to the step of pasteurizing/sterilizing the food.

FIG. 9 depicts the steps of this method applied to the liquid food as it passes through the tube, from its inlet to its outlet, in addition to the previous steps of sterilization, validation and provision to the system of the data obtained in the previous step of validation.

### DESCRIPTION OF THE INVENTION

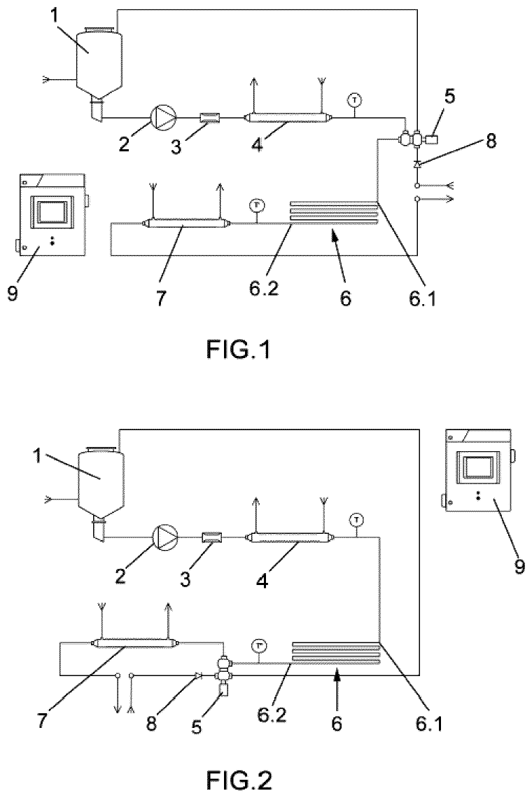

The present disclosure is schematically depicted in FIGS. 1 and 2. Said FIGS. 1 and 2 each shows a flowchart of the system used for the implementation of the steps of the method for reducing and/or eliminating a target agent from a fluid present in a liquid food, which is pumped through a tube by means of a fluid pump.

In this particular embodiment, the method also considers a reference treatment, said reference treatment being treatment F₀, widely known in the industry as an indicator of the cumulative case fatality of a microorganism.

Therefore, in a particular embodiment, the fatalities of the present method will be expressed against the reference treatment using its own parameters, T_{ref}=121.1° C. and Z=10° C. and against the target agent, or target microorganism, designated as Bacillus stearothermophilus using random parameters Dt=1000 seconds and Z=7.3° C.

Thus, both FIG. 1 and FIG. 2 show a system comprising a tank (1) containing the pumpable food to be treated by means of the corresponding heat treatment for the reduction of the target microorganism Bacillus stearothermophilus. The liquid food flow rate is pumped from the tank (1) by means of the pump (2) to a first heat exchanger (4), where its temperature will be increased up to a predetermined value by means of heat exchange with a heating fluid.

After the liquid food flow rate leaves the first heat exchanger (4), said flow rate is pumped until it enters the tube (6). A flow meter (3), located between the pump (2) and the heat exchanger (4), makes it possible to measure the flow rate Q of the pumpable food. Additionally, a temperature sensor measures the temperature T of pumpable food flow rate before it enters the tube (6), that is, before the fluid enters the tube (6) through its inlet (6.1).

Additionally, at the outlet of the tube (6), the pumpable food is introduced into a second heat exchanger (7), by means of which the temperature of said pumpable food is reduced through heat exchange with a cooling fluid. Previously, a temperature sensor measures the temperature T′ of the pumpable food flow rate after it exits from the tube (6) through its outlet (6.2).

After the cooling obtained in the second heat exchanger (7), the pumpable food, already treated in the interior of the tube (6), is packaged in packaging equipment.

Additionally, this system comprises a bypass valve (5), located in the case of FIG. 1 at the first end of the tube (6), while in FIG. 2 it is located at the second end of the tube (6). Both systems also include a non-return valve (8), which prevents diverting the pumpable food after the bypass valve (5) through undesired paths.

FIGS. 1 and 2 also show control and storage means (9), which make it possible to carry out the different steps of the present method, particularly those calculations necessary for the different steps.

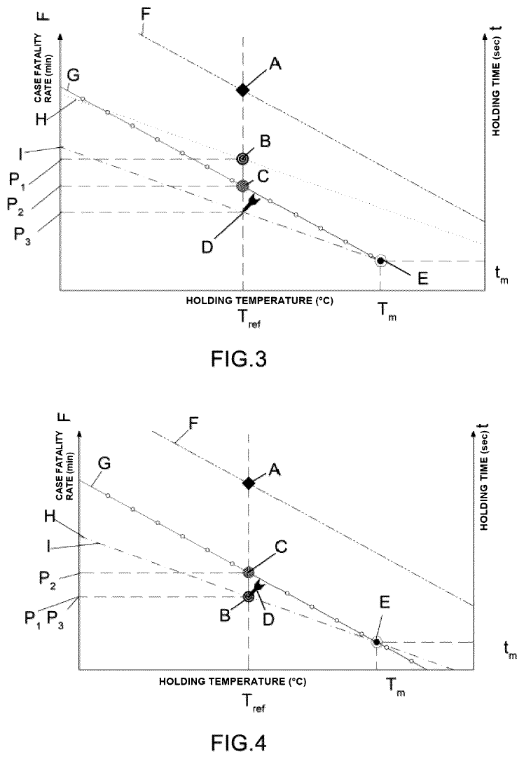

FIG. 3 shows a validation graph, where the value of the case fatality rate provided by the current treatment is lower than the value of the required target case fatality rate, that is, Fᵢ<F_{target}. This indicates that the process, and therefore its predetermined values, have not been validated.

As this figure shows, point B is the minimum required F value or P value against the target agent at the reference temperature T_{ref}, in order to meet the objective of safety and/or quality of the target agent from the present heat treatment, while point E represents the current pasteurization/sterilization treatment against the target agent and the reference agent, referenced with respect to holding temperature Tₘ and holding time tₘ.

In order for the validation criterion to be met, that is, for the value of the case fatality rate provided to be equal to the value of the target or required case fatality rate, that is, Fᵢ=F_{target}, it is necessary for said point B to coincide with point D, which represents the current treatment against the target agent, as depicted in FIG. 4.

In combination with these points, it is also necessary to take into account point D, which represents the F value or P value of the treatment equivalent to the current treatment at the reference temperature T_{ref}, as well as point C, which represents the F value or P value provided by the system at the reference temperature T_{ref} against the reference agent.

Additionally, point A represents the unitary reference treatment (equal to one minute), that is, the unitary F value or P value.

Finally, the points of intersection with the vertical axis, that is, with the value of the case fatality rate, represent the following:
P₁: threshold fatality required against the target agent,
P₂: threshold fatality against the reference agent, and
P₃: fatality provided against the target agent.

These points are also present in FIGS. 4 to 7.

FIG. 4 shows, for the same validation graph, that point B and point D coincide. In this case, the process, and therefore its predetermined values, have been validated, showing said coincidence.

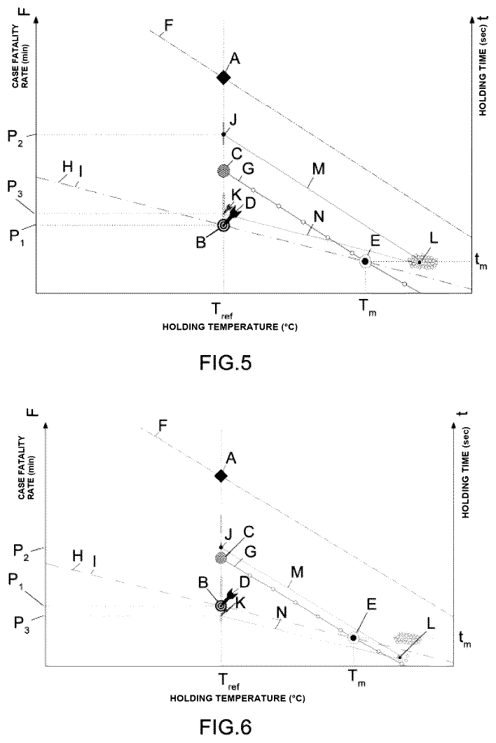

FIG. 5 shows a particular embodiment of a graphic verification and control process of the system, where the values of the case fatality rates provided against the target agent (Fi) and against the reference agent (Fi′) are represented.

It can also be observed in this particular embodiment that the value of the case fatality rate provided against the target agent (Fi) exceeds the value of the required target case fatality rate F_{target}, already obtained in the previous validation process, shown in FIGS. 3 and 4, and where it can also be seen that the value of the case fatality rate provided against the reference agent (Fi′) is also higher than the value obtained in the previous validation step F_{target}.

Graphically, the points depicted in FIG. 5 are the following:
Point D: represents the F value or P value of the treatment equivalent to the current treatment at the reference temperature T_{ref} against the target agent,
Point K: represents the current record of the F value or P value of the treatment equivalent to the current treatment at the reference temperature T_{ref} against the target agent,
Point C: represents the F value or P value provided by the system at the reference temperature T_{ref} against the reference agent,
Point J: represents the current record of the current F value or current P value of the system at the reference temperature T_{ref} against the reference agent,
Point E: represents the pasteurization/sterilization treatment validated against the target agent and the reference agent, and
Point L: represents the current pasteurization/sterilization treatment against the target agent and the reference agent.

Additionally, FIG. 5 shows the following points:
Point A: represents the F value or P value of the unitary reference treatment,
Line I: represents the treatments equivalent to the current treatment against the target agent,
Line G: represents the equivalent treatments against the current treatment against the reference agent,
Line H: represents the threshold line delimiting valid treatments against the target agent,
Line F: represents the line of reference treatments equivalent to the unitary one,
Line N: represents the line of treatments equivalent to the current treatment against the target,
Line M: represents the line of treatments equivalent to the current treatment against the reference agent.

In the case of FIG. 6, for another particular embodiment of the graphic verification and control process, the values of the case fatality rates provided against the target agent (Fi) and against the reference agent (Fi′) are observed, as is the fact that the value of the case fatality rate provided Fi is lower than the value of the required target case fatality rate F_{target} obtained in the previous validation process. The lines and points represented correspond to those mentioned in FIG. 5.

However, it can also be seen that the value of the case fatality rate provided against the reference agent in this case is higher than the value obtained in the validation. In this particular embodiment, the safety or quality of the food is not guaranteed and the system would act on the controls to regulate the heating means and/or the flow rate until achieving Fi≥Fo.

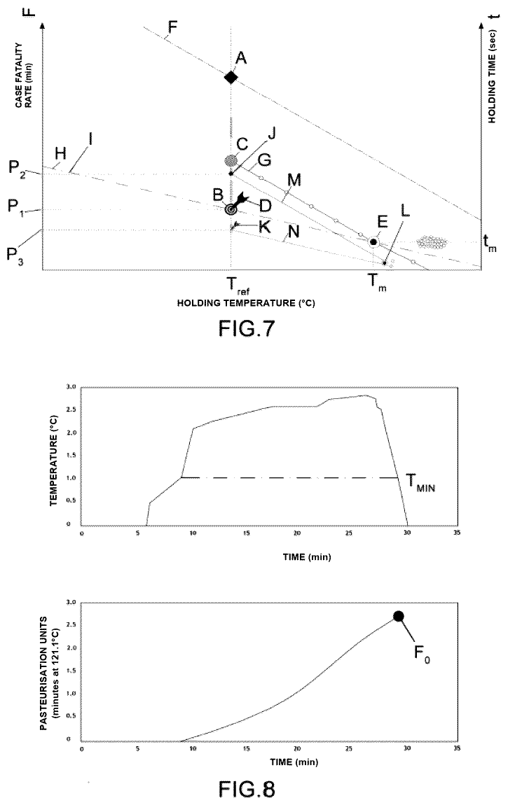

Lastly, FIG. 7, relating to a particular embodiment of a graphic verification and control process, represents the values of the case fatality rates provided against the target agent (Fi) and against the reference agent (Fi′), while it is also observed that the value of the case fatality rate provided Fi is lower than the value of the required target case fatality rate F_{target} obtained in the previous validation process.

Additionally, this FIG. 7 shows that the value of the case fatality rate provided against the reference agent in this case does not exceed the value obtained in the validation either. In this particular embodiment, the safety or quality of the food is not guaranteed and the system would act on the controls to regulate the heating means and/or the flow rate until achieving Fi≥Fo. The lines and points represented correspond to those mentioned in FIGS. 5 and 6.

FIG. 8 shows an example of the graph of the sterilization process prior to the step of pasteurizing/sterilizing the food.

Two graphs can be seen in this figure, the upper one relating to the evolution of the temperature of the fastest particle of the food over time, where the minimum temperature for which a sterilizing effect of the system is obtained after applying the sterilization step is also shown in broken line.

Additionally, the lower graph shows the evolution of the cumulative fatality provided by the system, in cumulative pasteurization units provided from the start of the treatment until completing the total holding time. This evolution is shown by the continuous line present in the graph.

The same graph in turn shows a point that graphically determines the fatality of the pasteurization treatment, or F₀.

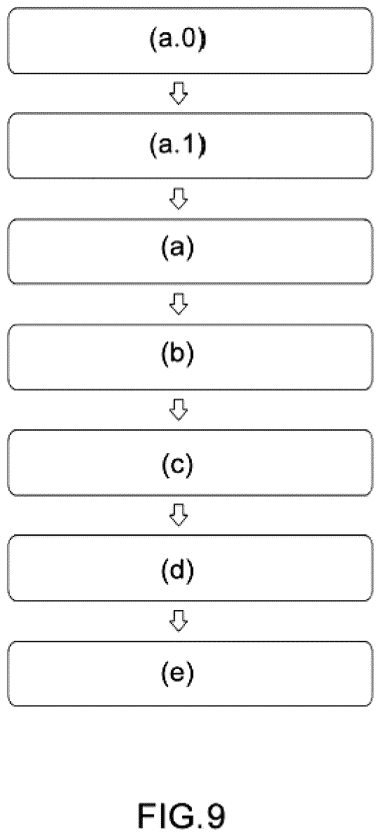

As can be observed in FIG. 9, the flowchart shown indicates the steps of the method applied to a liquid food as it passes through the tube, from its inlet to its outlet.

In this case, prior to the entry of the liquid food into the tube, step a.0) of sterilizing the tube is carried out, using steam or hot water. Said flow rate of steam or hot water is maintained at a temperature higher than the threshold value T_{start sterilization} for a total time tₘᵢ, during which time the flow of steam or hot water is recirculating throughout the system, that is, in the interior volume of the tube to reduce its population of microorganisms once the preset or target value F₀ has been reached, reaching the desired level of sterilization in the interior volume of said tube and in the cooling means after the tube, if any.

Next, the system performs a step a.1) of validating the variables required for the treatment, these being:
t_{validated}: the holding time of the fluid=28.3 seconds.
T_{validated}: the temperature of the fluid at the outlet of the tube=133.5° C.
F_{target}: the value of the required target case fatality rate=1413.66 seconds.

Subsequently, and once the values of these variables have been validated, step a) is carried out, wherein they are provided to the storage means of the control means, so that the treatment carried out on the liquid food inside the tube is based on these initial parameters, previously validated.

The system has temperature probes located at the inlet and/or outlet of the tube, which allow the recording of the temperature of the fluid Tᵢ for the instants of time t considered.

In the present embodiment, the system has a temperature probe at the outlet of the tube.

In this way, step b) of the method makes it possible, by means of the actuation of the probe at the outlet of the tube, to record in the storage means of the control means, the different values of the temperature of the fluid Tᵢ for the instants of time t considered. In addition, the method makes it possible, by means of the records coming from the flow rate probe, to calculate the holding time tₘᵢ of the fluid in the interior volume V of the tube, wherein:

$${t}_{mi} = \frac{{V}_{M}}{{Q}_{i}}$$

In the present example, the value of the volume necessary in the previous expression, based on the dimensions of the system, is Vₘ=0.00785398 m³.

Said holding time tₘᵢ includes the calculation of the holding time factored by a magnification factor FM considering the circulation regime of the food, thus establishing a conservative criterion for determining the heat treatment to be applied. In the present particular embodiment, the fastest food particle is considered for each of the records of the flow rate value Qᵢ for each instant tᵢ.

The density and viscosity values used to calculate the FM in this exemplary embodiment are the following:

$$Density = 1.13 kg / l$$

$$Viscosity = 236 cP$$

Flow behaviour index (n)=1

Reynolds number (Re) obtained: 16.93

Based on said temperature records of the fluid Tᵢ and of the holding time of the fastest particle t_{mi′}, the control means of the system make it possible, in a step c) of the present method, to calculate the value of the case fatality rate of the target agent provided, Fᵢ, for each of the instants of time t considered.

In particular, the value of the case fatality rate provided, Fᵢ, is calculated based on the following ratio for the target agent, by the control means:

$${F}_{i} = {\int }_{{t}_{0}}^{{tm}_{i}} 1 {0}^{( \frac{{T}_{i} - {T}_{ref}}{Z} )} dt$$

Where T_{ref}=121.1° C. and Z=7.3° C.

On the other hand, the value of the case fatality rate provided compared to the reference agent, F′ᵢ, is calculated based on the following relationship by the control means:

$${F}_{i}^{′} = {\int }_{{t}_{0}}^{{tm}_{i}} 1 {0}^{( \frac{{T}_{i} - {T}_{ref}}{Zref} )} dt$$

Where T_{ref}=121.1° C. and Z=10° C.

Once the value of the fatality rate provided, Fᵢ, at each instant of time t is known, the system allows the comparison of said values with the value of the required target case fatality rate, F_{target}, previously determined in step a.1).

In this way, step d) of the present method carries out the comparison of values, checking if Fᵢ<F_{target} by means of the control methods.

In the event that the previous condition is met, the treatment carried out is not adequate to achieve the target value of reduction of the population of the target microorganism or target agent, so the control means carry out modifications in the configuration of the system to, thus, modify the parameters of the applied treatment, treatment temperature and/or flow rate or holding time and increase its efficacy on liquid food.

In this way, when step d) of the method is verified, that is, when the condition Fᵢ<F_{target} is verified, the control means could act on the bypass valve of the system, so that the inlet flow liquid food flow rate in the interior of the tube or at its outlet is diverted and returned to the previous storage tank from where the pump that circulates the fluid food is fed.

Thus, with these modifications of the parameters of the temperature and flow rate system, there are obtained additional records of the temperature of the fluid Tᵢ and holding time tₘᵢ for the instants of time t considered, and therefore modified values of the value of the case fatality rate provided Fᵢ, until the condition is verified Fᵢ≥F_{target}, which indicates that the applied treatment has the required efficacy.

In this particular embodiment, a series of two records are considered to evaluate the value of the case fatality rate in various situations:

| | | | | | | Number of |
|---|---|---|---|---|---|---|
| | | | | | Fatality supplied | log-decimal |
| Flow | | Fastest particle | Fatality required | Fatality supplied | against the | reductions of |
| rate Qᵢ | Temperature | residence time | against target | against target | reference agent | target agent |
| (l/h) | Tᵢ (° C.) | (s) | agent F_{target} (s) | agent (s) | (s) | n_{target agent} |
| 355 | 137 | 39.823005468 | 1413.660307597 | 6000.897727348 | 1549.294693642 | 8.4898723 |
| 420 | 132.2 | 33.659921288 | 1413.660307597 | 1115.991896979 | 433.623785099 | 1.5788685 |

1-11. (canceled)

12. A method for reducing and/or eliminating a target agent from a fluid by means of a system comprising:
a tube extending between a first end and a second end, forming an internal volume V according to a path,
wherein the first end is a fluid inlet and the second end is a fluid outlet,
wherein the fluid enters the internal volume V of the tube through the inlet of the tube and runs along the path to the outlet of the tube, wherein said fluid is driven by a pump,
heating means fluidically connected to the inlet of the tube,
at least one fluid flow rate sensor, configured to measure the flow rate Q of the fluid driven by the pump,
at least one temperature sensor, located at the first and/or second end of the tube, configured to measure the temperature T of the fluid driven by the pump,
control means, comprising a controller and storage means,
the method comprising the following steps:
a) providing the storage means of the control means with the following predetermined values:
t_{validated}, holding time of the fluid in the internal volume V of the tube, from its inlet to its outlet,
T_{validated}, temperature of the fluid at the outlet of the tube,
F_{target}, value of the case fatality rate required for the reduction and/or elimination of the target agent,
b) introducing and circulating by means of the pump, during the holding time t_{validated}, the fluid at the temperature T_{validated} in the interior volume V of the tube,
c) recording, by means of the at least one temperature sensor and the at least one flow rate sensor, for each instant tᵢ, ∀i=0, 1, . . . , m, the real temperature value of the fluid Tᵢ, and the real flow rate value Qᵢ, of the fluid in the interior volume V of the tube,
d) calculating for each instant of time tᵢ, using the control means and based on the records of the real temperature Tᵢ and the real flow value Qᵢ of the fluid recorded in step c), the holding time tₘᵢ of the fluid in the interior volume V of the tube, wherein:
$${t}_{m i} = \frac{V}{{Q}_{i}}$$
e) calculating for each instant tᵢ, using the control means, the value of the case fatality rate provided, Fᵢ, according to the values recorded and calculated in steps c) and d), and wherein:
$${F}_{i} = {\int }_{{t}_{0}}^{{t}_{m i}} 1 {0}^{( \frac{{T}_{i} - {T}_{ref}}{Z} )} dt ,$$
wherein Z is a kinetic property of the reduction and/or elimination of the target agent and T_{ref} the chosen reference temperature
f) verifying, for each instant tᵢ, using the control means, if the condition Fᵢ<F_{target} is met, and
g) regulating, using the control means, if the condition of step f) is met, the real temperature value of the fluid Tᵢ and/or the real flow rate value Qᵢ of the fluid in the interior volume V of the tube, which verifies the condition Fᵢ≥F_{target}.

13. The method for reducing and/or eliminating a target agent from a fluid according to claim 12, wherein step d) further comprises calculating the holding time factored by a magnification factor FM based on the circulation regime of the fluid.

14. The method for reducing and/or eliminating a target agent from a fluid according to claim 12, wherein step e) further comprises calculating the value of the case fatality rate provided against the reference agent, Fᵢ′, according to the values recorded and calculated in steps c) and d), and wherein:
$${F}_{i}^{′} = {\int }_{{t}_{0}}^{{t}_{mi}} 1 {0}^{( \frac{{T}_{i} - {T}_{ref}}{Z} )} dt$$
wherein Z is a specific value of the reference agent used to define the reference treatment and T_{ref} is the chosen reference temperature with respect to which the case fatality rate, Fi′ is to be expressed.

15. The method for reducing and/or eliminating a target agent from a fluid according to claim 12, wherein step g) is carried out by means of a PID algorithm of the controller of the control means.

16. The method for reducing and/or eliminating a target agent from a fluid according to claim 12, comprising a step a.1) of validating the values of step a).

17. The method for reducing and/or eliminating a target agent from a fluid according to claim 16, wherein step a.1) is carried out based on a predetermined validation graph.

18. The method for reducing and/or eliminating a target agent from a fluid according to claim 12, wherein the verification step f) is carried out by means of a predetermined verification graph.

19. The method for reducing and/or eliminating a target agent from a fluid according to claim 12, further comprising the previous step of sterilizing the tube of the system, reducing and/or eliminating a target agent present in said tube by means of a sterilizing fluid, preferably by means of steam or hot water.

20. The method for reducing and/or eliminating a target agent from a fluid according to claim 19, wherein the previous step of sterilizing the tube comprises the steps of:
i. providing the storage means of the control means with the following predetermined values:
F_{validatedsterilization}: Value of the target sterilization index,
T_{start sterilization}: temperature value in at least one given sensor from which the cumulative sterilization index is calculated,
ii. introducing the sterilizing agent, preferably steam or hot water, into the interior volume V of the tube, and causing it to circulate by means of the pump, until reaching the temperature T_{start sterilization},
iii. recording, by means of the at least one temperature sensor, the real temperature Tᵢ for an instant of time tᵢ,
iv. storing, in the storage means, the value of the real temperature Tᵢ recorded in iii) for each instant of time tᵢ
v. calculating the cumulative case fatality rate F₀ according to the ratio:
$${F}_{0} = {\int }_{{t}_{0}}^{{t}_{m i}} 1 {0}^{( \frac{{T}_{i} - {T}_{ref}}{Zref} )} dt$$
wherein Z_{ref} is a reduction and/or elimination kinetic property of the reference agent and T_{ref} the chosen reference temperature with respect to which the case fatality rate F₀ is to be expressed,
vi. determining if the value of the cumulative case fatality rate F₀ and the value of the validated sterilization index F_{validatedsterilization}, meet the ratio F₀>F_{validatedsterilization}, that is, the cumulative case fatality rate is higher than the value of the target sterilization index established in step i).

21. The method for reducing and/or eliminating a target agent from a fluid according to claim 12, wherein the system further comprises a bypass valve, and wherein step g) of the method further comprises acting on the opening and/or closing of the bypass valve.

22. A computer program configured to implement the method according to claim 12 when it is run on the controller of the control means of the system as defined in claim 12.
